(19) United States
(12) Patent Application Publication
(10) Pub. No.: US 2022/0409125 A1
(43) Pub. Date: Dec. 29, 2022

(54) BIO-SIGNAL ACQUISITION AND FEEDBACK

(71) Applicant: ASHVA WEARABLE TECHNOLOGIES PRIVATE LIMITED, Hyderabad Telangana (IN)

(72) Inventors: Anmol SAXENA, Bangalore Karnataka (IN); Sandeep Reddy GOLUGURI, Hyderabad Telangana (IN)

(21) Appl. No.: 17/756,272

(22) PCT Filed: Nov. 20, 2020
(86) PCT No.: PCT/IN2020/050973

§ 371 Date: May 20, 2022

(30) Foreign Application Priority Data
Nov. 20, 2019 (IN) 201941047363

(51) Int. Cl. A61B 5/00; A61B 5/11; G16H 10/60; G16H 50/50

(52) U.S. Cl. CPC A61B 5/4585; A61B 5/6828; A61B 5/11; A61B 5/7203; A61B 5/0022; G16H 10/60; G16H 50/50

(57) ABSTRACT

Computing devices, wearable devices, networked environments, and methods for bio-signal acquisition and monitoring skeletomuscular parameters are provided. First data indicative of movement parameters of knee joint of a patient for a test type is obtained from a wearable device. Second data indicative patient-specific parameters of the patient are obtained. A cluster to which the patient belongs is identified based on the patient-specific parameters and a prediction model. A normative range for the test type for the cluster is determined. The first data is processed to determine whether the patient falls within the normative range of the cluster and result is provided.

### TECHNICAL FIELD

The present subject matter relates in general to bio-signal acquisition and feedback, and in particular, to devices, systems, and methods for bio-signal acquisition and feedback.

### BACKGROUND

Degenerative joint diseases, such as arthritis, ankylosing spondylitis, and the like, generally, occur due to breakdown and wear of cartilage which cushions bones at the joint. Further, other joint traumas, such as, fracture, dislocations, and the like, may also occur, for example, due to accidents, sports injuries, and the like. Generally, on diagnosis of degenerative joint diseases and joint trauma, rehabilitative techniques, such as physiotherapy and the like, can be provided by a healthcare professional. Degenerative joint diseases and joint trauma are diagnosed using expensive techniques, such as myography, X-ray, computer tomography (CT), bone scanning, ultrasonography, and the like. The same techniques are also, generally, used to monitor prognosis and progress of rehabilitation in the affected joint.

### BRIEF DESCRIPTION OF DRAWINGS

The detailed description is described with reference to the accompanying figures. In the figures, the left-most digit(s) of a reference number identifies the figure in which the reference number first appears. The same numbers are used throughout the drawings to reference like features and components.

FIG. 1 illustrates an example networked environment for bio-signal acquisition and feedback, in accordance with an implementation of the present subject matter.

FIG. 2(a) illustrates a block diagram of a wearable device for bio-signal acquisition and feedback, in accordance with an example implementation of the present subject matter.

FIG. 2(b) illustrates a schematic of a wearable device for bio-signal acquisition and feedback, in accordance with an example implementation of the present subject matter.

FIGS. 3(a)-(c) illustrate an example wearable device coupled to a knee joint for bio-signal acquisition and feedback, in accordance with an example implementation of the present subject matter.

FIGS. 4(a)-4(b) illustrate an example compression sock placed on a knee joint for bio-signal acquisition and feedback, in accordance with an example implementation of the present subject matter.

FIGS. 5(a)-5(b) illustrate an example compression sock placed on a knee joint for bio-signal acquisition and feedback, in accordance with an example implementation of the present subject matter.

FIG. 6 illustrates an example computing device for bio-signal acquisition and feedback, in accordance with an example implementation of the present subject matter.

FIG. 7 illustrates an example method for bio-signal acquisition and feedback, in accordance with an example implementation of the present subject matter.

### DETAILED DESCRIPTION

The present subject matter provides devices, networked environments, and methods which can be used for bio-signal acquisition and feedback. The devices and methods can be used for diagnosis, prognosis, and monitoring progress of rehabilitation in patients with joint trauma and degenerative joint disorders.

Conventionally, diagnosis, prognosis, and rehabilitation of joint trauma is monitored manually, for example, by a physiotherapist or a doctor, using expensive techniques, such as myography, X-ray, computer tomography (CT), bone scanning, ultrasonography, and the like. Further, monitoring also involves usage of expensive devices, such as goniometers, isokinetic dynamometers, and the like. Thus, unlike blood tests for diagnosis and monitoring of infectious diseases and deficiency disorders, there is no integrated, portable, and accessible system for assessment of skeletomuscular injuries, measuring the effectiveness of rehabilitative therapy, and predicting or extrapolating prognosis of the joint trauma.

Due to the lack of such accessibility, accuracy of diagnosis and prognosis relies on the competency of the physical therapist or the doctor. If they are not skilled, they may not be able to identify the root cause of pain or trauma, and may, thus, misdiagnose the injury, thereby, reducing effectiveness of any subsequent treatment plan. Further, during treatment, in the absence of adequate monitoring techniques, reduced patient compliance to rehabilitative therapy is observed as there is no benchmark available to visibly see progress. Non-compliance may cause an aggravation of existing skeletomuscular injuries or may lead to re-injuries.

The present subject matter addresses these and other problems and provides networked environments, computing devices, wearable devices and methods for acquiring information of skeletomuscular health. The devices of the present subject matter are low-cost, portable, and have wearable form factor. The computing devices and methods of the present subject matter work in consonance with the wearable devices to receive information of skeletomuscular health, analyze the information, and provide data to healthcare providers and the patient for diagnosis, monitoring prognosis, rate of rehabilitation, effectiveness of therapy, and the like.

An example networked environment of the present subject matter includes a wearable device and a computing device. The wearable device can be adapted to be associated with a knee joint of a patient to receive bio-signals corresponding to skeletomuscular data. The wearable device comprises a plurality of sensors to measure first data corresponding to movement parameters of the knee joint of the patient for a test type. The movement parameters include bio-mechanical and physiological assessment parameters of the knee joint.

In one example, the wearable device of the present subject matter can comprise at least a pair of electronic cores for attachment to the knee joint of the patient. Each electronic core can comprise an electrode and a plurality of sensors, such as Inertial Motion Unit (IMU) sensors, surface electromyography sensors, pressure sensors, force sensors, and the like. The wearable device can further comprise a processing unit, for example, a microprocessor, to receive signals from the plurality of sensors corresponding to the first data and process the signals to remove unwanted noise. A device memory may also be provided to store raw data corresponding to the signals. The wearable device of the present subject matter may be in the form of, for example, a joint brace, compression socks, and the like.

The wearable device further comprises a wireless communication module, hereinafter referred to as communication module, to transfer signals corresponding to the first data to the computing device for analysis of the first data over a communication network. The signals transferred by the communication module may be the raw signals or signals processed to remove unwanted noise. In one example, the networked environment comprises other devices, such as user devices of the patient and/or the healthcare provider, to provide second data indicative of patient-specific parameters of the patient and to receive the analysed results from the computing device.

In one example, the computing device can receive the first data obtained from the wearable device over the communication network and the second data indicative of the patient-specific parameters of the patient from, for example, from the user devices. Based on the received first data and the second data, the analysis module can identify a cluster to which the patient belongs based on the patient-specific parameters. A normative range for a test type for the cluster can be determined. The first data can then be processed by the analysis module to determine whether the patient falls within the normative range of the cluster to which belongs for the test type. The computing device may implement various techniques, such as Machine Learning (ML) or Artificial Intelligence (AI) based techniques, to analyse the first data and the second data to diagnose and monitor the patient. The computing device can provide a result of whether the patient falls within the normative range for that cluster for the test type. The result may be provided, for example, on the user device of a physiotherapist or a doctor or the patient. Accordingly, treatment options may be determined and effectiveness of treatment over time may also be monitored.

Thus, the present subject matter helps to effectively and accurately assess the health of joints. Further, progress of rehabilitation, prediction and extrapolation of health of the joint in the future, and the like, can be monitored in an inexpensive manner. As the device is portable, it may also be used for home physiotherapy care, at clinics, at hospitals, at chiropractic clinics, traditional treatment centres, for example, acupuncture, acupressure, naturopathy, myofascial release, and any other therapies used for rehabilitation of skeletomuscular injuries.

The above and other features, aspects, and advantages of the subject matter will be better explained with regard to the following description and accompanying figures. It should be noted that the description and figures merely illustrate the principles of the present subject matter along with examples described herein and, should not be construed as a limitation to the present subject matter. It is thus understood that various arrangements may be devised that, although not explicitly described or shown herein, embody the principles of the present disclosure. Moreover, all statements herein reciting principles, aspects, and examples thereof, are intended to encompass equivalents thereof. Further, for the sake of simplicity, and without limitation, the same numbers are used throughout the drawings to reference like features and components.

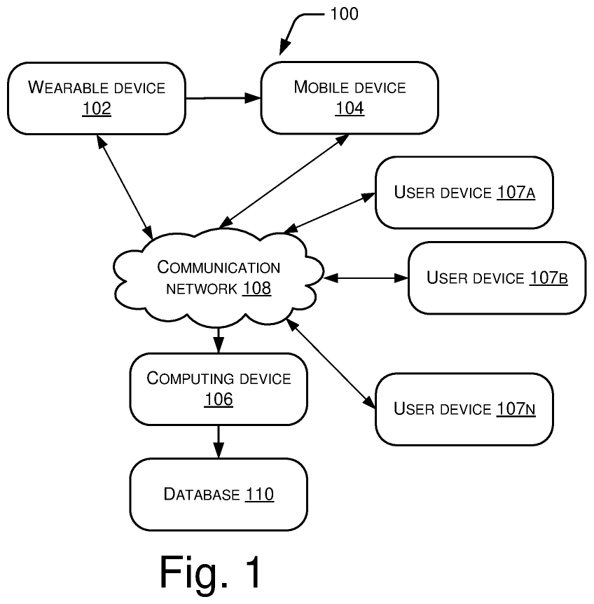

FIG. 1 illustrates a block diagram of a networked environment 100 for bio-signal acquisition and feedback, in accordance with an example implementation of the present subject matter. The networked environment 100 can comprise a wearable device 102 communicatively connected to a mobile device 104 and a computing device 106. The networked environment 100 can further include a plurality of user devices 107a, 107b . . . 107n, hereinafter referred to as user devices 107. In one example, the mobile device 104 may also be a user device 107. The computing device 106, mobile device 104, and the user devices 107 may individually be, for example, a smartphone, a smartwatch, a tablet, a laptop, a personal computing device, or the like. The wearable device 102 and the mobile device 104 can communicate with each other over a short range communication network (not shown) in one example and with the computing device 106 over a communication network 108. Further, the computing device 106 can also communicate with the user devices 107 over the communication network 108. In some examples, two or more of the mobile device 104, computing device 106, and user device 107 may be integrated with each other and may thus function as a single device instead of separate devices. In another example, the computing device 106 may be implemented over a cloud network.

The communication network 108 may be a wireless network, a wired network, or a combination thereof. The communication network 108 can also be an individual network or a collection of many such individual networks, interconnected with each other and functioning as a single large network, e.g., the Internet or an intranet. The communication network 108 can include different types of networks, such as intranet, local area network (LAN), wide area network (WAN), the internet, and such. The communication network 108 may also include individual networks, such as, but not limited to, Global System for Communication (GSM) network, Universal Telecommunications System (UMTS) network, Long Term Evolution (LTE) network, etc. Accordingly, the network includes various network entities, such as base stations, gateways, servers, and routers; however, such details have been omitted to maintain the brevity of the description.

The wearable device 102 can be a portable device which can be adapted to be associated with a knee joint of a patient. The wearable device 102 can help in collecting bio-signals, hereinafter also referred to as signals, corresponding to the movement of the knee joint of the patient. The term bio-signals refers to signals associated with a joint portion of the patient and includes signals consonant with movement of the joint portion, such as, acceleration, gravity vector, quaternion parameters, crepitus sound, and the like.

To collect the signals, the wearable device 102 can comprise a plurality of sensors. The plurality of sensors measures first data corresponding to movement parameters of the knee joint of the patient. The movement parameters comprise both the bio-mechanical and physiological assessment parameters of the knee joint. The plurality of sensors may be selected from inertial motion units (IMUs), surface electromyography (sEMG) sensors, skin pressure sensors, force sensors, and combinations thereof. The wearable device 102 may also comprise, for example, an amplifier or signal processing module to amplify the signals captured by the plurality of sensors and a noise filtering module to process the signals to remove unwanted noise.

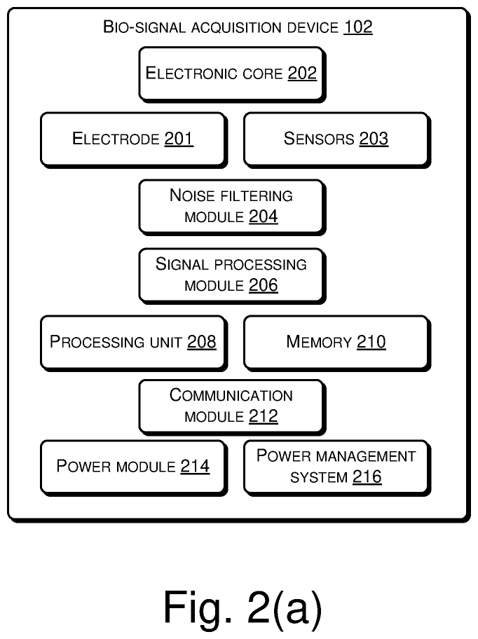

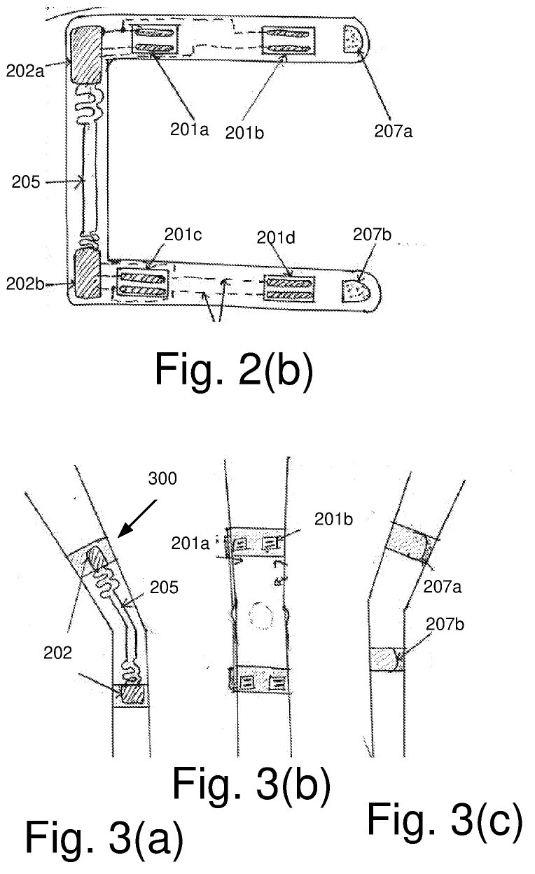

In one example, the wearable device 102 may also provide any preliminary alerts to the patient in case of any emergencies. For example, based on a preliminary analysis by a processing unit provided in the wearable device 102, the wearable device 102 can detect if the patient is doing some exercise incorrectly or that they can modify their position or movement to assume a better stance. Based on this detection, feedback can be provided by the wearable device 102 to the patient. The feedback may be visual or auditory or both. An example wearable device 102 is explained later with reference to FIG. 2(a).

The signals corresponding to the first data, without any processing or processed signals, obtained by the wearable device 102 can be communicated to the mobile device 104. The signals can be communicated to the mobile device 104 by using a communication module of the wearable device 102 as will be explained later with reference to FIG. 2(a).

The mobile device 104 or the wearable device 102 can further communicate the signals to the computing device 106 over the communication network 108 for analyzing the signals to provide monitoring, diagnosis, and prognostic related results.

In addition to the first data, second data corresponding to patient-specific parameters of the patient can also be provided to the computing device 106, for example, by using the mobile device 104, a user device 107, or combinations thereof. The patient-specific parameters may be selected from age, weight, height, gender, occupation, lifestyle habits, hereditary information, past medical data, length, girth, height of the limbs, kind of footwear, geographical location, type of bedding, and combinations thereof.

The networked environment 100 can also comprise a database 110 coupled to the computing device 106 to receive and store the raw signals or processed signals. In FIG. 1, the database 110 is shown external to the computing device 106. It is to be understood that the database 110 may also be internal to the computing device 106.

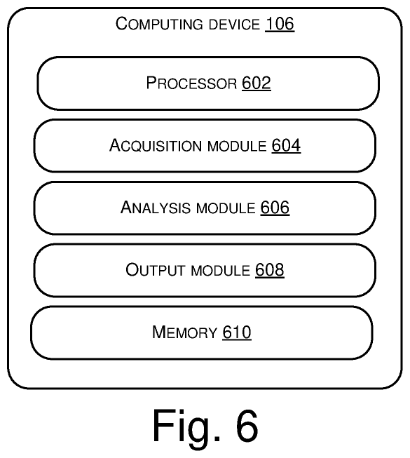

The computing device 106 may comprise a processor and modules executable by the processor. In operation, the computing device 106 can analyse the first data and the second data to determine whether the patient's parameters for the test type fall within the normative range of a cluster to which the patient can be classified based on the patient-specific parameters. For this, the computing device 106 may use prediction models developed for each test type using machine learning techniques. By offloading the analysis to the computing device 106, higher accuracy of diagnosing and monitoring recovery can be achieved. Further, by using machine learning to train the prediction model using data from healthy volunteers for each test type, higher accuracy of joint health assessment and prognosis can be achieved. Various aspects of the functioning of the computing device 106 are explained later with reference to FIG. 6.

FIG. 2(a) illustrates a block diagram of an example wearable device 102 for bio-signal acquisition and feedback, in accordance with an example implementation of the present subject matter. The wearable device 102 may be adapted to be associated with a knee joint of a patient. The wearable device 102 can comprise an electronic core 202. In one example, the wearable device 102 can comprises two or more electronic cores to monitor and receive signals from the knee joint.

In one example, the wearable device 102 includes two electronic cores, where each electronic core 202 may be positioned on an upper and a lower part of the knee joint, for example, by using adhesive patches, Velcro straps, and the like. The two electronic cores may be connected to each other via wires, cables or wirelessly through Bluetooth, beacons, WiFi, and the like. In another example, a single long or smaller two-piece electronic core which has a flexible PCB can be stuck on the skin, for example, around the knee joints, laterally of the lower limbs, or anteriorly. The electronic core 202 may be associated with electrodes 201 and a plurality of sensors 203 to measure first data corresponding to movement parameters of the knee joint of the patient for a test conducted corresponding to a particular test type. The test type may be selected from joint range of motion, muscle strength, proprioception, balance test, gait analysis, lifestyle monitoring, type of activity, impact shock, muscle endurance, muscle stamina, time to full recover, return to sport, degree of disability, degree of functionality, flight ration, smoothness index, pronation index, rotation index, coordination index, range index, pain index, system index, and combinations thereof. The same wearable device 102 may be used to perform tests for obtaining movement parameters for different test types. Accordingly, before a test is performed, an input indicative of the test type may be provided to the wearable device 102, for example through the mobile device 104 or user device 107 or an input interface)not shown) on the wearable device 102.

The plurality of sensors 203 may be selected from inertial motion units (IMUs), surface electromyography (sEMG) sensors, skin pressure sensors, force sensors, and combinations thereof. The first data may depend on the type of sensors. For example, when the sensor is an IMU, the first data corresponds to roll or pitch or yaw, linear acceleration, angular acceleration, muscular vibration, environmental vibrations, gravity vectors, quaternions, rotational matrix, motion of the testing ground, and the like; when the sensor is the surface electromyography (sEMG) sensors, the first data corresponds to magnitude of muscular activation and time duration of muscular activation; and when the sensors is a pressure or force sensor, the first data corresponds to muscular activation, time duration of muscular activation, force of limb movement, shear force of limb movement and the like.

The signals measured by the plurality of sensors 203 may be processed in the wearable device 102. To process the signals measured by the plurality of sensors 203, the wearable device 102 can comprise a noise filtering module 204 to filter noise from the measured signals and a signal processing module 206 to amplify the signals. Processing of signals, for example, by filtering and amplification, is known in the art and the same has not been described herein for the sake of brevity.

The signals or the processed signals may then be received by a processing unit 208. The processing unit 208 may be implemented as one or more microprocessors, microcomputers, microcontrollers, digital signal processors, central processing units, state machines, logic circuitries, and/or any devices that manipulate signals based on operational instructions. Among other capabilities, the processing unit 208 fetches and executes computer-readable instructions stored in a memory 210. The functions of the various elements shown in the figure, including any functional blocks labeled as processor, may be provided through the use of dedicated hardware as well as hardware capable of executing computer readable instructions.

The processing unit 208 can perform a preliminary analysis of the signals or pre-signals and provide analysis output to the patient, for example, by using a display module (not shown) in the wearable device 102. The signal, processed signal, and the preliminary analysis may be stored in the memory 210. The memory 210 may serve as a repository for storing data that may be fetched, processed, received, or created by wearable device 102 or received from connected devices, such as mobile device 104, user devices 107, and the like. While the memory 210 is shown as internal to the wearable device 102, it will be understood that the memory 210 may be external to the wearable device 102 and can be accessed by the wearable device 102 using various communication means.

Additionally, the wearable device 102 may include various interfaces, memories, other data, and the like, which are not shown for brevity. The interfaces may include a variety of computer-readable instructions-based interfaces and hardware interfaces that allow interaction with other communication, storage, and computing devices, such as network entities, web servers, databases, and external repositories, and peripheral devices. The memories may include any non-transitory computer-readable medium including, for example, volatile memory (e.g., RAM), and/or non-volatile memory (e.g., EPROM, flash memory, etc.). The memories may include an external memory unit, such as a flash drive, a compact disk drive, an external hard disk drive, or the like. The other modules may include modules for operation of the wearable device 102, such as operating system, and other applications that may be executed on the wearable device 102. Other data may include data used, retrieved, stored, or in any way manipulated by the wearable device 102.

The wearable device 102 also comprises a communication module 212. The communication module 212 can help in communicating the signals or processed signal to the mobile device 104 or the computing device 106 for further analysing as previously explained. The wearable device 102 further comprises a power module 214 and a power management system 216. In one example, the power module 214 may include a rechargeable battery and the power management system 216 can monitor the status of the battery and provide indication when the battery is to be recharged.

As previously explained, the wearable device 102 may be incorporated, for example, in a joint brace, a compression sock, and the like. FIG. 2(b) depicts an example schematic of the wearable device 102, in accordance with an implementation of the present subject matter. The wearable device 102 as shown in FIG. 2(b), depicts two electronic cores 202a, 202b connected by wire 205. The electronic core 202a is coupled to a first pair of electrodes 201a, 201b and the electronic core 202b is coupled to a second pair of electrodes 201c, 201d. The electronic cores 202a, 202b, the first pair of electrodes 201a, 201b, and the second pair of electrodes 201c, 201c may be associated with a flexible brace structure which can be coupled to the joint portion of the patient by Velcro 207. However, other mechanisms for coupling to the joint portion, for example, by using adhesive pads may be used. The electronic core 202a and the first pair of electrodes 201a, 201b may be coupled to an upper part of the joint portion while the electronic core 202b and the second pair of electrodes 201c, 201d may be coupled to a lower part of the joint portion. This is further explained with reference to FIGS. 3(a)-3(c).

FIGS. 3(a)-(c) illustrate an example joint brace 300 coupled to a knee joint, in accordance with an implementation of the present subject matter. FIG. 3(a) depicts a lateral view of the knee joint with the electronic cores 202a and 202b, depicted collectively as 202, provided above and below the knee joint, respectively. FIG. 3(b) illustrates a front view of the knee joint showing the first pair of electrodes 206a and 206b provided above the knee joint and the second pair of electrodes 206c and 206b provided below the knee. FIG. 3(c) illustrates a medial side view of the knee joint showing the Velcro 207a, 207b for coupling the joint brace 300 to the knee.

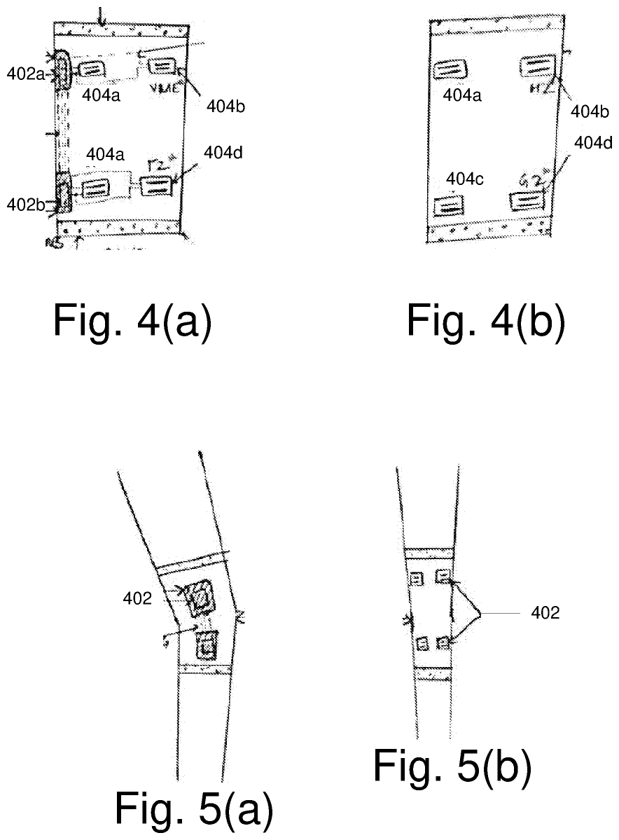

While FIG. 3(a)-3(c) depict a joint brace coupled to the knee joint, the wearable device 102 may be implemented in other wearable forms as well. For example, the wearable device 102 may also be incorporated and used in compression socks. FIG. 4(a) depicts an anterior view of an example compression socks. The compression socks depict two electronic cores 402a and 402b. Each core is associated with a pair of electrodes, i.e., electronic core 402a is associated with pair of electrodes 404a, 404b and the electronic core 402b is associated with pair of electrodes 404c, 404d. The electronic core 402a and electronic core 402b may be electrically coupled to each other via wires, cables, and the like, as previously explained. FIG. 4(b) illustrates posterior view of the compression socks depicting the first pair of electrodes 404a, 404b and the second pair of electrodes 404c, 404d.

FIGS. 5(a)-5(b) illustrates an example compression sock placed on a knee joint, in accordance with an implementation of the present subject matter. FIG. 5(a) depicts a lateral view of the compression socks showing the electronic cores 402a, 402b, shown collectively as 402. FIG. 5(b) depicts a posterior view of the compression shows depicts the first pair of electrodes 404a, 404b and the second pair of electrodes 404c, 404d, shown collectively as 404. In one example, as shown in FIG. 5(a), the electronic core 402a and 402b may be mounted in a core holder and may be detachable.

In operation, with reference to FIGS. 2(a), 2(b), 3(a)-(c), 4(a)-(b), 5(a)-(b), the wearable device 102 can be coupled to, i.e., positioned on or around, the knee joint of the patient. On coupling the wearable device 102, the wearable device 102 may be calibrated. To calibrate the wearable device 102, the limb may be kept in a near-to-stationary position and the plurality of sensors 203 may be allowed to calculate an average initial vector. The average initial vector forms a reference point or a reference plane. In one example, the stability of the limb may also be determined during calibration. For example, if the average initial vector is not stable, then the wearable device 102 may alert the patient (or the physical therapist or the doctor) that the plurality of sensors 203 are not correctly positioned or that the patient is unstable, such as, in case of tremors or shivering in the adjoining muscle or the knee joint.

Further, prior to collecting the signals corresponding to the movement of the knee joint, second data corresponding to patient-specific parameters may be collected by and/or provided to the computing device 106. The patient-specific parameters may be selected from age, weight, height, gender, occupation, lifestyle habits, hereditary information, past medical data, length, girth, height of the limbs, kind of footwear, geographical location, type of bedding, and combinations thereof. The second data may be provided, in one example, using a user interface on the wearable device 102. In another example, the second data may be collected and provided using the mobile device 104 or the user device 107. In one example, the second data collected from the patient may be provided to the computing device 106 over the communication network 108.

On calibration, the plurality of sensor 202b can measure first data corresponding to movement parameters of the knee joint of the patient. In one example, the communication module 212 can transfer signals corresponding to the first data from the wearable device 102 to the computing device 106 or to the mobile device 104 which may then transfer it to the computing device 106. In another example, the signals may first be processed in the wearable device 102, for example, by the noise filtering module 204 to remove unwanted noise and the signal processing module 206 to amplify the signals. In said example, the wearable device 102 can transfer the processed signals to the computing device 106 or to the mobile device 104 which then sends it to the computing device 106. In another example, the signals or the processed signals may also be provided to the processing unit 208 which can perform a preliminary analysis and provide alerts to the patient, the physical therapist or the doctor. The first data and the second data may then be together analyzed by the computing device 106.

FIG. 6 illustrates an example computing device 106 for bio-signal acquisition and feedback, in accordance with an implementation of the present subject matter. The computing device 106 comprises a processor 602, an acquisition module 604, an analysis module 606, an output module 608, and the memory 610.

The processor(s) 602 may be implemented as one or more microprocessors, microcomputers, microcontrollers, digital signal processors, central processing units, state machines, logic circuitries, and/or any devices that manipulate signals based on operational instructions. Among other capabilities, the processor(s) 602 fetches and executes computer-readable instructions stored in the memory 610. The functions of the various elements shown in the figure, including any functional blocks labeled as processor, may be provided through the use of dedicated hardware as well as hardware capable of executing computer readable instructions.

The acquisition module 604, the analysis module 606, the output module 608, collectively referred to as modules, may be coupled to the processor 602 and may be executable by the processor 602, and may include, amongst other things, routines, programs, objects, components, data structures, and the like, which perform particular tasks or implement particular abstract data types. The modules may be implemented as hardware, software, or a combination of the two.

The memory 610 may serve as a repository for storing data that may be fetched, processed, received, or created by computing device 106 or received from connected devices. While the memory 610 is shown as internal to the computing device 106, it will be understood that the memory 610 may be external to the computing device 106 and can be accessed by the computing device 106 using various communication means.

Additionally, the computing device 106 may include various interfaces, memories, other data, and the like, which are not shown for brevity. The interfaces may include a variety of computer-readable instructions-based interfaces and hardware interfaces that allow interaction with other communication, storage, and computing devices, such as network entities, web servers, databases, and external repositories, and peripheral devices. The memories may include any non-transitory computer-readable medium including, for example, volatile memory (e.g., RAM), and/or non-volatile memory (e.g., EPROM, flash memory, etc.). The memories may include an external memory unit, such as a flash drive, a compact disk drive, an external hard disk drive, or the like. The other modules may include modules for operation of the computing device 106, such as operating system, and other applications that may be executed on the computing device 106. Other data may include data used, retrieved, stored, or in any way manipulated by the computing device 106.

The acquisition module 604 can receive the first data and the second data. As previously explained, the first data can be obtained from a wearable device, for example, wearable device 102, over a communication network, for example, communication network 108. The first data is indicative of movement parameters of the knee joint to which the wearable device 102 is coupled for obtaining parameters of a test corresponding to a test type. The movement parameters comprise the bio-mechanical and physiological assessment parameters of the knee joint.

For example, the first data may be roll or pitch or yaw, linear acceleration, angular acceleration, muscular vibration, environmental vibrations, gravity vector, quaternions, rotational matrix, motion of the testing ground, muscle activation, magnitude of muscle activation, time period of muscle activation, force of the limb movement, shear force on limbs, and the like. The test type is one of: joint range of motion, muscle strength, proprioception, balance test, gait analysis, lifestyle monitoring, type of activity, impact shock, muscle endurance, muscle stamina, time to full recover, return to sport, degree of disability, degree of functionality, flight ration, smoothness index, pronation index, rotation index, coordination index, range index, pain index, system index, and combinations thereof.

The second data is indicative of patient-specific parameters of the patient. The second data may be obtained, for example, from the wearable device 102, the mobile device 104, the user device 107, or a combination thereof. The patient-specific parameters may be age, weight, height, gender, occupation, lifestyle habits, hereditary information, past medical data, length, girth, height of the limbs, kind of footwear, geographical location, type of bedding, and combinations thereof.

In one example, on receiving the first data, the computing device 106 can process the first data. For this, the computing device 106 can comprise a pre-processing module (not shown). The pre-processing module may also be executable by the processor 602 to process the first data, for example, to filter noise.

The first data and the second data may be analyzed by the analysis module 606. The analysis performed by the analysis module 606 may be based on a prediction model generated by, for example, machine learning and artificial intelligence techniques. The prediction model defines a plurality of clusters to which the patient may be identifiable. In one example, the prediction model may be generated by a module of the computing device 106. In another example, the prediction model may be generated external to the computing device 106 and fed to the computing device 106 for implementation.

To generate the prediction model, population data (also referred to as training data) comprising a plurality of patient specific parameters and test results or movement parameters for various tests corresponding to the different test types as mentioned above, may be received. The population data may be obtained from healthy volunteers who do not have any problem in the knee joints so that the normative ranges corresponding to healthy joints may be determined The patient-specific parameters may be selected from age, weight, height, gender, occupation, lifestyle habits, hereditary information, past medical data, length, girth, height of the limbs, kind of footwear, geographical location, type of bedding, and combinations thereof. The patient specific parameters and the movement parameters for each test type may be fed to an ML/AI engine (not shown) for identifying the clusters and normative ranges.

In one example, the ML/AI engine may first identify the significant parameters from among the patient specific parameters for each test type. The significant parameters may be those that have a significant correlation with the movement parameters. For this, in one example, for a test type, the ML/AI engine may perform a regression between the patient specific parameters and the movement parameters to identify weights for each patient specific parameter for that test type. The parameters that have weights with a magnitude above a threshold value may be considered to be significant. In one example, a first set of parameters may be found to be significant for a first test type whereas a second set of parameters may be found to be significant for a second test type.

Further, the data may be clustered by the ML/AI engine based on the movement parameters and identified significant parameters to create clusters for each test type. The clusters may be identified based on centroids of the movement parameters (test results) using techniques known in the art. For example, for a first test type, it may be identified that males of age less than 30 and Body Mass Index (BMI) of 21-25 have similar test results and hence form one cluster, while males of age less than 30 and BMI 26-30 form another cluster. In one example, considering age ranges of less than 30, 31-40, 41-50, 51-60, and greater than 61; gender groups of male, female, and others; and BMI ranges of 21-25, 26-30, 31-35, and 36-40, the number of clusters formed may be 60 (5 age ranges*3 gender groups*4 BMI ranges). In another example, for another test type, the clusters formed may be different as the age groups or the significant parameters identified may be different.

Thus, the population data can be analyzed to identify the plurality of clusters for each test type. Further, from the test results for each data point in a cluster, the normative range can be found for that cluster. The normative range may be defined by, for example, an average value of the test result for that cluster and the standard deviation or variance. Thus, the prediction model may be trained by the ML/AI engine using population data to create clusters and identify the normative range for each of the clusters for each test type.

Based on the prediction model, the analysis module 606 can analyze the first data and the second data received by the computing device 106. For this, the analysis module can identify a cluster from the plurality of clusters to which the patient belongs based on the second data, i.e., the patient-specific parameters. The identification may be based on the similarity of the patient-specific parameters with characteristics of the significant parameters which defines the cluster.

On identifying the cluster, the analysis module 606 can determine a normative range for the test type for that cluster. For example, when the test type is muscle strength, the analysis module 606 can determine the normative range for muscle strength for the cluster to which the patient has been identified as belonging.

The first data may be processed by the analysis module 606 to determine whether the patient falls within the normative range of the cluster to which the patient belongs for the test type.

In one example, for a test type, only a first set of parameters from the first data may be relevant. For example, for joint range of motion, the first set of parameters of roll or pitch or yaw, gravity vector, quaternions, and rotational matrix may be relevant. In said example, based on test type, the analysis module 606 can derive the first set of parameters from the first data and process the same to obtain a final value for the test type. The final value may be processed to determine whether the patient falls within the normative range of the cluster to which the patient belongs for the test type. It will be understood that training data may also be processed in a similar manner to generate the prediction model.

In another example, on deriving the first set of parameters, intermediate values may be determined for intermediate test parameters. For example, for joint range of motion, the intermediate test parameters may be flexion and extension lag which may dependent on the first set of parameters roll or pitch or yaw, gravity vector, quaternions, and rotational matrix. The intermediate values for the intermediate test parameters to obtain the final value for the test type can be processed. The final value may be processed to determine whether the patient falls within the normative range of the cluster to which the patient belongs for the test type. It will be understood that training data may also be processed in a similar manner to generate the prediction model.

The various first data obtained from different sensors of the wearable device 102 are provided in Table 1.

TABLE 1
Sensor and first data

| | | | Pressure or |
|---|---|---|---|
| | IMU | sEMG | force sensor |
| | (1) | (2) | (3) |
| A | Roll/Pitch/Yaw | Muscle | Muscle |
| | | activation | activation |
| B | Linear | Magnitude of | Magnitude of |
| | Acceleration | muscle | muscle |
| | | activation | activation |
| C | Angular | Time period | Force of the |
| | Acceleration | of muscle | limb |
| | | activation | movement |
| D | Muscular | | Shear force on |
| | Vibration | | limb |
| E | Environmental | | |
| | vibration | | |
| F | Gravity vector | | |
| G | Quaternion | | |
| H | Rotational | | |
| | matrix | | |
| I | Motion of the | | |
| | testing ground | | |

The various intermediate test parameters that can be derived using the first data listed in Table 1 are listed in Table 2.

TABLE 2
Intermediate test parameters derived based on first data

| | | First set of parameters of first data |
|---|---|---|
| | | relevant |
| | Intermediate | (1, 2, 3 - represent sensor; a-h represent |
| | test parameter | rows; in Table 1) |
| i | Flexion | 1a + 1f + 1g + 1h |
| ii | Extension Lag | 1a + 1f + 1h |
| iii | Limb | 1a + 1d + 1f |
| | shivering | |
| iv | Activity | 1a + 1b + 1c + 1d + 1f + 1g + 1h + 1i + 2a or 3a |
| | Tempo | |
| v | Pronation | 1a + 1f + 1h + 1i |
| | excursion | |
| vi | Varys or | 1a + 1f + 1h + 1i |
| | Valgus Static | |
| vii | Varys or | 1a + 1c + 1f + 1g + 1h + 1i |
| | Valgus | |
| | Dynamic | |
| viii | Muscle fatigue | 2a + 2b + 2c OR 3a + 3b + 3c |
| ix | Maximum | 2a + 2b + 2c OR 3a + 3b + 3c |
| | Voluntary | |
| | Contraction | |
| x | Stride length | 1a + 1b + 1c + 1f + 1g + 1h + 1i + 2a or 3a |
| xi | Contact time | 1a + 1f + 1g + 1h + 2a or 3a + 2b or 3b + 1c or |
| | | 2c |
| xii | Flight time | 1a + 1f + 1g + 1h + 2a or 3a + 2b or 3b + 1c or |
| | | 2c |
| xiii | No of steps | 1a + 1f + 1g |
| xiv | Stride rate | xiii + camera inputs |
| xv | Propulsion | 1a + 1c + 1f + 1g + 1h + 2a or 3a + 2b or 3b |
| | force | |
| xvi | Braking force | 1a + 1c + 1f + 1g + 1h + 2b or 3b + 2c or 3c |
| xvii | Q angle | 1a + 1f + 1g + 1h or 5 |
| xviii | Distance | x + xiii |
| | travelled | |
| xix | Gait velocity | xviii per minute |
| xx | Pronation | 1a + 1c + 1f + 1g + 1h |
| | velocity | |

The final value of the test type may be obtained directly based on the first set of parameters or based on intermediate values obtained from the first set of parameters. The test type and the first set of parameters and the intermediate test parameters that they depend on are provided in Table 3.

TABLE 3
Test type and dependencies

| | Dependency on first data and |
|---|---|
| | intermediate test parameters (Refer |
| Test type | Table 1 and Table 2) |
| Joint Range of | i + ii |
| Motion | |
| Muscle Strength | i + ii + iii + iv + viii + ix + ankle weights |
| | data |
| Proprioception | i + ix |
| Balance | i + iii + vi + viii + ix |
| Gait Analysis | Σxvi:xvi |
| Functional | All second derivatives |
| Assessment and | |
| Lifestyle | |
| Monitoring | |
| Type of Activity | x + xi + xii + xiii + ix |
| Impact shock | i + ii + iv + viii + xv + ix + xvi |
| Muscle endurance | iv + i + viii + ix |
| Muscle stamina | viii + ix + Muscle strength |
| Time to full | Extrapolation of all test parameters |
| recovery | to normative range |
| Return to sport | Extrapolation of test result to |
| | normalcy for Impact shock + |
| | Muscle strength + Muscle |
| | endurance + Muscle Stamina + |
| | Coordination Index + Gait analysis + |
| | Functional Assessment and |
| | Lifestyle Monitoring + |
| | Proprioception |
| Degree of | Joint range of motion + Muscle |
| disability | Strength + Pain Score + |
| | Proprioception + Balance + Functional |
| | Assessment and Lifestyle |
| | Monitoring |
| Degree of | 1-Degree of disability |
| functionality | |
| Flight ratio | xi/xii |
| Smoothness index | xi/xvi or xvi/xv |
| Pronation index | v/xxviii |
| Rotation index | 1a or 1h |
| Coordination index | Balance/2a or 3a |
| Range index | I |
| Force index | Muscle strength + 3d + 3e |
| Pain index | patient-specific paramaters + i + viii + ix |
| System index | Σxvii:xxv |

Some example test types are also described below based on the Tables 1, 2, and 3:

In joint range of motion, the intermediate test parameters are flexion and extension lag (refer Table 2 and 3). The intermediate test parameters depend, in turn on roll or pitch or yaw, gravity vector, quaternions, and rotational matrix (refer Table 2 and 1). Thus, based on roll or pitch or yaw, gravity vector, quaternions, and rotational matrix, the analysis module 606 can determine intermediate values for intermediate test parameters, namely, flexion and extension lag, which may then be used to obtain the final value for joint range of motion. The analysis module 606 can compare or process the final value with the normative range for the cluster to which the patient is identified to determine whether the patient falls within the normative range of the cluster.

In the case of balance tests, the intermediate test parameters are flexion, limb shivering, varys or valgus static, muscle fatigue and maximum voluntary contraction (refer to Table 2 and 3). The first data based on which these intermediate test parameters can be determined are provided in Table 1 and 2. The analysis module 606 can compare or process the final value with the normative range for the cluster to which the patient is identified to determine whether the patient falls within the normative range of the cluster.

In the case of joint proprioception, the intermediate test parameters are flexion and maximum voluntary contractions, the values of which can be determined based on Table 2 and Table 1. The values of the intermediate test parameters may be used to determine the final value for joint proprioception. The analysis module 606 can compare or process the final value with the normative range for the cluster to which the patient is identified to determine whether the patient falls within the normative range of the cluster.

In one example, the test type may also include the intermediate test parameter. By using the acceleration, gravity vector, quaternions, force/pressure on the joint, and surface electromyography, the stride length may be derived. The number of steps depends upon an angle between upper and lower bones of the knee joint in the sagittal plane of the patient's body. The acceleration between the start and stop of each step should ideally be equal to zero. However, due to inherent noise of the accelerometer, a deviation is seen from zero. This deviation is taken as error count. The error count can be deducted from the acceleration over each sample of the step cycle and, thereby, double integrated to calculate the stride length. Comparison of the stride length will be drawn between injured knee and uninjured knee to assess the degree of injury, analyse time for full recovery, to help doctors assess the amount of rehabilitation further required.

Number of steps can also be determined. The initial relative angle when the person is standing is taken as 0 degrees, with respect to gravity. The angles vary when the person is in motion. The analysis module can check the positions in which the relative angles become zero with respect to gravity. Two such instances can record one step. Based on the stride length and number of steps, the distance traveled can be computed as a product of the stride length and number of steps. The stride rate can be calculated as number of steps per minute. Further, contact time (time the foot remains on the ground during gait) and flight time (time during which the foot remains in air during gait) can be computed. For this, data from both needs may be measured and analysed at the same time. The acceleration, knee flexion, and angular velocity can be compared in each step and each limb. The limb for which the duration of acceleration and angular velocity is recorded to be lesser in comparison to the other limb is recorded as the contact time for that limb and flight time for the other limb.

The instantaneous acceleration at the start and end of each step can also be computed. The acceleration spike can be recorded at the start and end of each step. The acceleration spike can be compared with a global average (which is calculated from same parameter dataset from healthy patients).

Impact shock, for example, on the ankle joint, measured at the heels, toes, and mid foot), braking force (on the foot, measured at the ankle joint), and propulsion force (force with which the body propels into the next step can also be computed based on signals obtained from the sEMG or pressure sensors. Average or cumulative force can be measured on the heel, toes, and midfoot by simple addition or mean deviation. For impact shock, the average or cumulative force at the heel, toes, and midfoot is calculated when the foot just touches the ground. Braking force is calculated by taking the average or cumulative force at the heel, toes, and midfoot when the other leg is in flight. Propulsion force is calculated by measuring the force used by the muscles to propel the body forward.

The active time may also be computed based on variation in the acceleration and knee flexion. Pronation excursion may also be computed based on distance of the limb movement in the coronal plane of the human body which is calculated similar to stride length calculation, as explained above, in the sagittal plane. The rotation of the knee joint can also be measured based on relative quaternions.

Muscle activation time can be computed based on standard deviation of the normal signal versus the activated muscle signal is compared to identify the muscle activation. The duration for which this standard deviation is measured is recorded as the muscle activation duration. The range of motion can be computed based on relative quaternions or Euler angles and gravity vector in each plane of the patient's body. The total muscle force used may be calculated based on inverse kinematics formulae. Balance of the patient can also be assessed, for example, by using a conventional Timed Up and Go test. Parameters like rotation of the limb, pronation excursion, range of motion, and stride length can provide the symmetrical movement of each limb with respect to each other to assess balance. Conventional staircase climbing test can be incorporated as a time-based assessment tool into the system.

Muscle strength of the limbs can also be assessed. Different weights can be tied to the lowest end of the lower limb, i.e. the ankle Based on the weight, maximum number of repetitions, the muscles that activate, frequency of muscle activation along with the test subject personal parameters like age, gender, BMI, occupation, angular velocity, range of motion, limb flexion and torque etc., the analysis module 606 calculates the muscle strength of the patient.

For monitoring rehabilitation, the final value obtained for each test type and the intermediate values for the intermediate test parameters may be stored in the memory 610 for each time the tests are conducted. The analysis module 606 can retrieve such historical data to determine if treatment process is working. This is further described with examples below:

Muscle endurance can be assessed. The patient can be asked to perform some exercises for the maximum number of repetitions possible. The amount of time the patient performs repetitions continuously is recorded for that limb and compared to the patient's historical data (which may be store in memory 610) as well as compared against the healthy limb to measure the muscle endurance improvement (in ratio, percentage, or any other relative format)

Muscle stamina may also be assessed. The patient can be asked to perform a high intensity activity. The amount of time for which the patient performs the high intensity activity is recorded as muscle stamina duration. The patient's historical data and healthy limb muscle stamina is compared to the time duration to calculate the improvement in muscle stamina.

Based on the analysis performed by the analysis module 606, the output module 608 can provide a result of whether the patient falls within the nominative range for that cluster for that test time. In one example, the output module 608 can also provide virtual reality (VR), augmented reality, 3D simulation, infographic, visual, and printed reports to the mobile device 104 or the user devices 107. For example, prior to measuring first data by the wearable device 102, a photograph or video of he patient while performing static or dynamic activity by using either a mobile phone camera or a specific camera. The same may be transmitted to the computing device 106.

In another example, an image of the patient standing straight is taken to determine the angle between hip and knees, knees and ankle, shoulders and hip, shoulder and elbow, elbow and wrist (i.e. the Q angle). The output module 608 can use an image processing technique to render a simulation of the patient.

In another example, a video may be taken by a trained personal while the patient performs certain exercises/tasks such as:
a. walking, jogging, running, or likewise—to analyze the gait and/or calibrate the wearable device with readings and inferences from video graphic gait analysis.
b. climbing up and down (a stair or a slope/incline plank) (for example: to determine the functional motion of the patients, to analyze the pelvic angles versus knee range of motion for assessment of ease of motion for the patient during daily activities like climbing a bus or stairs, etc.)

In one example, an overall disability score may be computed for the patient based on an aggregate of test results from multiple test types. For example, in case for a particular test type, the test result is within normative range, a score of 1 may be provided for that test type, else a score of zero may be provided. In another example, various scores between 1 and 0 may be provided depending on how much the test result deviates from the normative range. The scores from all the test types of interest may be summed to determine an overall score for the patient. Further a percentage disability score may be arrived at based on the maximum score that may be obtained. For example, if results from 5 test types are considered and the score obtained by a patient is 3 while maximum score that can be obtained in 5, the patient may be given a disability score of 40% (i.e., (1−(3/5))*100). Further effectiveness of a physiotherapy or treatment protocol may be determined based on the variation in the disability score over time. For example, if the disability score reduces over time, it can indicate that the physiotherapy protocol is successful and can be continued with. The disability score may also e provided as an output by the output module 608.

Based on result provided by the output module 608 and/or simulation provided to the user devices 107 and the mobile device 104, the healthcare provider (such as physical therapist or doctor) can remotely monitor the patient.

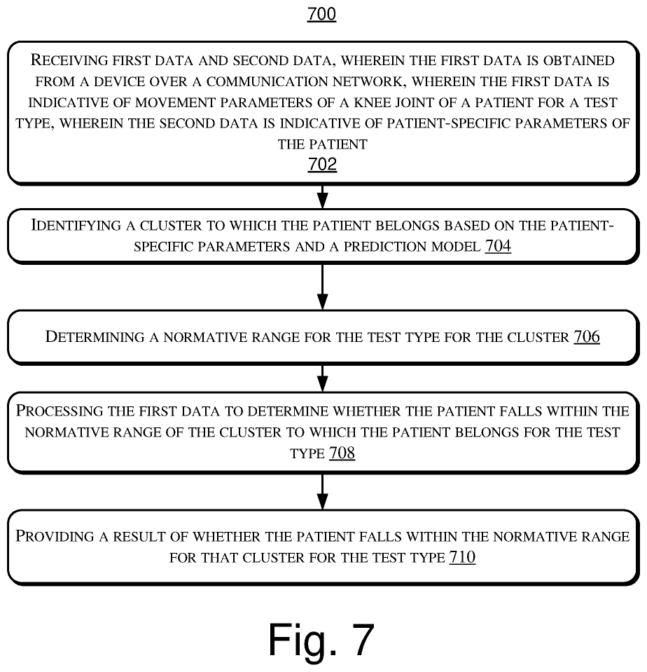

Thus, by implementing various aspects of the present subject matter, monitoring of joints of patients can be achieved using a portable, low-cost device and system. Further, based on this tele-rehabilitation can also be enabled based on the bio-signals acquired using the device. The methods used for the bio-signal acquisition and processing as discussed above will now be further described with reference to FIG. 7.

FIG. 7 illustrates an example method 700 for monitoring skeletomuscular parameters of a patient, in accordance with principles of the present subject matter. The order in which the method 700 is described is not intended to be construed as a limitation, and any number of the described method blocks can be combined in any order to implement method 700 or an alternative method. Additionally, individual blocks may be deleted from the method 700 without departing from the spirit and scope of the subject matter described herein. Furthermore, the method 700 may be implemented in any suitable hardware, computer readable instructions, firmware, or combination thereof. For discussion, the method 700 is described with reference to the implementations illustrated in FIGS. 1-6.

A person skilled in the art will readily recognize that steps of the method 700 can be performed by programmed computers. Herein, some examples are also intended to cover program storage devices and non-transitory computer readable medium, for example, digital data storage media, which are computer readable and encode computer-executable instructions, where said instructions perform some or all of the steps of the described method 700. The program storage devices may be, for example, digital memories, magnetic storage media, such as magnetic disks and magnetic tapes, hard drives, or optically readable digital data storage media.

At block 702, the method 700 comprises receiving a first data and a second data. The first data is indicative of movement parameters of a knee joint of a patient for a test type. The movement parameters comprise the bio-mechanical and physiological assessment parameters of the knee joint. The second data is indicative of patient-specific parameters. The first data and the second data may be received, for example, by the acquisition module 604 of the computing device 106. The first data may be obtained, for example, from a wearable device 102. The second data may be obtained, for example, from mobile device 104, the user devices 107, and combinations thereof.

At block 704, the method 700 comprises identifying a cluster to which the patient belongs based on the patient-specific parameter. In one example, the analysis module 606 identifies the cluster to which the patient belongs based on a prediction model. The prediction model may be generated based on population data comprising a plurality of types of data by analyzing the population data, and identifying, based on the population, the plurality of clusters, as discussed above.

At block 706, the method 700 comprises determining a normative range for the test type for the cluster. In one example, the normative range is determined by the analysis module 606 of the computing device 106.

At block 708, the method 700 comprising processing the first data to determine whether the patient falls within the normative range of the cluster to which the patient belongs for the test type. In one example, the analysis module 606 processes the first data to determine whether the patient falls within the normative range. In one example, a first set of parameters may be derived from the first data. The first set of parameters may be relevant to the test type. Based on the first set of parameters, intermediate values for intermediate test parameters are determined The intermediate values may be processed to obtain a final value for the test type. The final value can be processed to determine whether the patient falls within the normative range of the cluster to which the patient belongs for the test type.

At block 710, the method 700 comprises providing a result of whether the patient falls within the normative range for that cluster for the test type. The output module 608 may provide the result to the mobile devices 104, the user devices 107, or combinations thereof. In one example, the output module 608 may provide. along with the result, the intermediate values for the intermediate test parameters, the final value of the test type, and a disability score to the mobile devices 104, the user devices 107, or combinations thereof. In another example, the output module 608 may also provide a simulation of test for ease in remote visualization.

Thus, the present subject matter provides a low cost, accessible technique for diagnosis, monitoring and treatment of skeletomuscular disorders and injuries. The present subject matter can help in providing feedback to the patient and the healthcare provider regarding treatment and prognosis of disease or injury.

Although the subject matter has been described in considerable detail with reference to certain examples and implementations thereof, other implementations are possible. As such, the scope of the present subject matter should not be limited to the description of the preferred examples and implementations contained therein.

1. A computing device comprising:
a processor;
an acquisition module, executable by the processor, to receive first data and second data, wherein the first data is obtained from a wearable device over a communication network, wherein the first data is indicative of movement parameters of a knee joint of a patient for a test type, wherein the second data is indicative of patient-specific parameters of the patient;
an analysis module, executable by the processor, to:
identify a cluster to which the patient belongs based on the patient-specific parameters and a prediction model;
determine a normative range for the test type for the cluster; and
process the first data to determine whether the patient falls within the normative range of the cluster to which the patient belongs for the test type; and
an output module, executable by the processor, to provide a result indicative of whether the patient falls within the normative range for that cluster for the test type.

2. The computing device as claimed in claim 1, wherein the prediction model defines a plurality of clusters to which a patient is identifiable, wherein to generate the prediction model, an ML/AI engine is to:
analyze population data comprising training data corresponding to the patient specific parameters; and
identify, based on analysis of the population data, the plurality of clusters.

3. The computing device as claimed in claim 1, wherein the analysis module is to:
derive a first set of parameters from the first data, wherein the first set of parameters is relevant to the test type;
determine, based on the first set of parameters, intermediate values for intermediate test parameters;
process the intermediate values for the intermediate test parameters to obtain a final value for the test type; and
process the final value to determine whether the patient falls within the normative range of the cluster to which the patient belongs for the test type.

4. The computing device as claimed in claim 1, wherein the computing device comprises a pre-processing module, executable by the processor, to process the first data to filter noise.

5. The computing device as claimed in claim 1, wherein the patient specific parameters are selected from age, weight, height, gender, occupation, lifestyle habits, hereditary information, past medical data, length, girth, height of the limbs, kind of footwear, geographical location, type of bedding, and combinations thereof.

6. The computing device as claimed in claim 1, wherein the test type are one of: joint range of motion, muscle strength, proprioception, balance test, gait analysis, lifestyle monitoring, type of activity, impact shock, muscle endurance, muscle stamina, time to full recover, return to sport, degree of disability, degree of functionality, flight ration, smoothness index, pronation index, rotation index, coordination index, range index, pain index, system index, and combinations thereof.

7. A networked environment comprising:
a wearable device adapted to be associated with a knee joint of a patient, wherein the wearable device comprises:
a plurality of sensors to measure first data corresponding to movement of the knee joint of the patient; and
a communication module to transfer signals corresponding to the first data to a computing device for analysis of the first data;
a computing device comprising:
a processor;
an acquisition module, executable by the processor, to receive the first data and second data, wherein the first data is obtained from the wearable device over a communication network, wherein the first data is indicative of movement parameters of a knee joint of a patient for a test type, wherein the second data is indicative of patient-specific parameters of the patient;
an analysis module, executable by the processor, to:
identify a cluster to which the patient belongs based the patient-specific parameters and a prediction model;
determine a normative range for the test type for the cluster; and
process the first data to determine whether the patient falls within the normative range of the cluster to which the patient belongs for the test type; and
an output module, executable by the processor, to provide a result indicative of whether the patient falls within the normative range for that cluster for the test type.

8. The networked environment as claimed in claim 7, wherein the wearable device comprises a processor to:
receive signals corresponding to the first data from the plurality of sensors; and
process the signals to remove unwanted noise.

9. The networked environment as claimed in claim 7, wherein the plurality of sensors is selected from inertial motion units (IMUs), surface electromyography (sEMG) sensors, skin pressure sensors, force sensors, and combinations thereof.

10. The networked environment as claimed in claim 7 comprising a plurality of user devices, wherein the output module is to provide the result on at least a user device of the plurality of user devices.

11. The networked environment as claimed in claim 7, wherein the the prediction model defines a plurality of clusters to which a patient is identifiable, wherein to generate the prediction model, an ML/AI engine is to:
analyze population data comprising training data corresponding to the patient-specific parameters; and
identify, based on analysis of the population data, the plurality of clusters.

12. The networked environment as claimed in claim 7, wherein the analysis module is to:
derive a first set of parameters from the first data, wherein the first set of parameters is relevant to the test type;
determine, based on the first set of parameters, intermediate values for intermediate test parameters;
process the intermediate values for the intermediate test parameters to obtain a final value for the test type; and
process the final value to determine whether the patient falls within the normative range of the cluster to which the patient belongs for the test type.

13. A method implemented by a computing device, the method comprising:
receiving first data and second data, wherein the first data is obtained from a wearable device over a communication network, wherein the first data is indicative of movement parameters of a knee joint of a patient for a test type, wherein the second data is indicative of patient-specific parameters of the patient;
identifying a cluster to which the patient belongs based on the patient-specific parameter and a prediction model;
determining a normative range for the test type for the cluster;
processing the first data to determine whether the patient falls within the normative range of the cluster to which the patient belongs for the test type; and
providing a result indicative of whether the patient falls within the normative range for that cluster for the test type.

14. The method as claimed in claim 13, wherein the method comprises generating a prediction model, wherein the prediction model defines a plurality of clusters to which a patient is identifiable, wherein the generating comprises:
analyzing population data comprising training data corresponding to the patient specific parameters; and
identifying, based on analysis of the population data, the plurality of clusters.

15. The method as claimed in claim 13, wherein the method comprises:
deriving a first set of parameters from the first data, wherein the first set of parameters is relevant to the test type;
determining, based on the first set of parameters, intermediate values for intermediate test parameters;
processing the intermediate values for the intermediate test parameters to obtain a final value for the test type; and
processing the final value to determine whether the patient falls within the normative range of the cluster to which the patient belongs for the test type.
